(19) United States
(12) Patent Application Publication
(10) Pub. No.: US 2019/0093092 A1
(43) Pub. Date: Mar. 28, 2019

(54) RNA GUIDED ERADICATION OF VARICELLA ZOSTER VIRUS

(71) Applicant: Temple University - of the Commonwealth System of Higher Education, Philadelphia, PA (US)

(72) Inventors: Kamel Khalili, Bala Cynwyd, PA (US); Hassen Wollebo, Philadelphia, PA (US)

(21) Appl. No.: 16/099,098

(22) PCT Filed: Dec. 12, 2016
(86) PCT No.: PCT/US16/66134

§ 371 Date: Nov. 5, 2018

Related U.S. Application Data
(60) Provisional application No. 62/332,027, filed on May 5, 2016.

(51) Int. Cl. C12N 9/22; C12N 15/11; A61K 38/46

(52) U.S. Cl. CPC C12N 9/22; C12N 15/11; C12N 2320/32; C12N 2310/20; A61K 38/465

(57) ABSTRACT

Compositions that specifically cleave target sequences in Herpesviridae, for example Varicella zoster virus (VZV) include nucleic acids encoding a Clustered Regularly Interspaced Short Palindromic Repeat (CRISPR) associated endonuclease and a guide RNA sequence complementary to a target sequence in VZV. These compositions are administered to a subject for treating an infection or at risk for contracting a VZV infection.

### FIELD OF THE INVENTION

The present invention relates to compositions that specifically cleave target sequences in Varicella Zoster Virus (VZV). Such compositions, which include nucleic acids encoding a Clustered Regularly Interspaced Short Palindromic Repeat (CRISPR) associated endonuclease and a guide RNA sequence complementary to a target sequence in VZV, can be administered to a subject having or at risk for contracting a VZV infection.

### BACKGROUND

Varicella zoster virus (VZV) is an exclusively human virus that belongs to the α-herpesvirus family. VZV is present worldwide and is highly infectious. Primary infection leads to acute varicella or “chickenpox”, usually from exposure either through direct contact with a skin lesion or through airborne spread from respiratory droplets (Sawyer M H, et al. J Infect Dis. 1994; 169:91-94; Gnann J W Jr., et al. N Engl J Med. 2002; 347:340-346). After initial infection, VZV establishes lifelong latency in cranial nerve and dorsal root ganglia, and can reactivate years to decades later as herpes zoster (HZ) or “shingles” (Gilden D H, et al. N Engl J Med. 2000; 342:635-645.3). More than 90% of adults in the United States acquired the disease in childhood, while the majority of children and young adults have been vaccinated with the live virus vaccine (Gnann J W Jr., et al. N Engl J Med. 2002; 347:340-346; Marin M, et al. MMWR Recomm Rep. 2007; 56:1-40).

Reactivation and replication of latent VZV, often decades later, correlates with a decline in cell-mediated immunity, which occurs in the elderly or those who are immunocompromised (Weinberg et al., Journal of Infectious Diseases (2009) 200: 1068-77). In some patients, pain associated with HZ can persist for months or even years after the HZ rash has healed, a complication referred to as post-herpetic neuralgia (PHN).

### SUMMARY

Embodiments of the invention provide a composition for treatment of a viral infection. The composition includes a nuclease and a sequence-specific targeting moiety that targets the nuclease to viral nucleic acid in vivo or in vitro thereby causing the nuclease to cleave the viral nucleic acid without interfering with host nucleic acid. In certain embodiments, the nuclease is a Cas endonuclease and the sequence-specific binding module comprises a guide RNA that specifically targets a portion of a viral genome. The Cas endonuclease and the guide RNA may be co-expressed in a host cell infected by a virus. In embodiments, the virus is a herpesvirus, for example Varicella zoster virus (VZV).

Methods and compositions of the invention may be used to deliver a CRISPR/gRNA/Cas complex to a cell (including entire tissues) that is infected by a herpesvirus, e.g. VZV. The CRISPR/gRNA/Cas complexes of the invention can be delivered by viral, non-viral or other methods to effectuate transfection. CRISPR/gRNA/Cas complexes are preferably designed to target viral genomic material and not genomic material of the host. In some embodiments, the targeted viral nucleic acid is associated with a virus that causes latent infection. Latent viruses may be, for example, Epstein-Barr virus, human cytomegalovirus, human herpesviruses 6 and 7, herpes simplex virus types 1 and 2, varicella-zoster virus, measles virus, or human papovaviruses.

Other aspects are described infra.

### BRIEF DESCRIPTION OF THE DRAWINGS

FIG. 1A is a schematic diagram of VZV genome with nucleotide number depicting VZV-ORF63 gene with motif1 and motif2 gRNA target sites.

FIG. 1B shows the sequence of the VZV genome at each of the two targeted sites (red highlight).

FIG. 2 shows the sequence of the VZV ORF 63. The underlined sequences show the forward and reverse primers. The ORF 63 motif1 (FM1) and motif2(FM2) sequences are shown in italics (red).

FIG. 3 shows the sequence of the VZV ORF 63. The sequences (blue) show the forward and reverse primers. The ORF 63 motif1 (FM1), motif2 (FM2) and motif3 (FM3) sequences are shown in italics (red).

FIG. 4A is a schematic diagram of VZV genome with nucleotide numbers showing VZV ORF 63 gene with motif 1, 2, and 3 gRNA target sites.

FIG. 4B shows the sequence of the VZV genome at each of the targeted sites (red highlight).

FIGS. 5A, 5B show that CRISPR/Cas9 introduces InDel mutations in the VZV ORF63 gene. FIG. 5A is a schematic representation of the VZV ORF63 genomic sequence. The positions and nucleotide compositions of ORF63m1, ORF63m2 and ORF63m3 targets including the PAM sequences (marked in red) are shown. The cutting site of SpCas9 is also indicated according with the positions of the three gRNAs. The positions and the sequence of primers (Fw and Rev) used in the PCR amplification are illustrated. FIG. 5B depicts a gel analysis of DNA fragments amplified by primers Fw and Rev in the ORF63-stable TC620 oligodendroglioma cell line transient transfected with combinations of px260-SpCas9-gRNA m1, m2, and m3 targeting VZV ORF63. The positions of the expected 752 bp amplicon and smaller DNA fragments of 693 bp, 592 bp, and 651 bp caused by cleavage of the ORF63 genomic sequence by using gRNA combinations of ORF63m1+ORF63m2, ORF63m1+ORF63m3 and ORF63m2+ORF63m3, respectively, are shown.

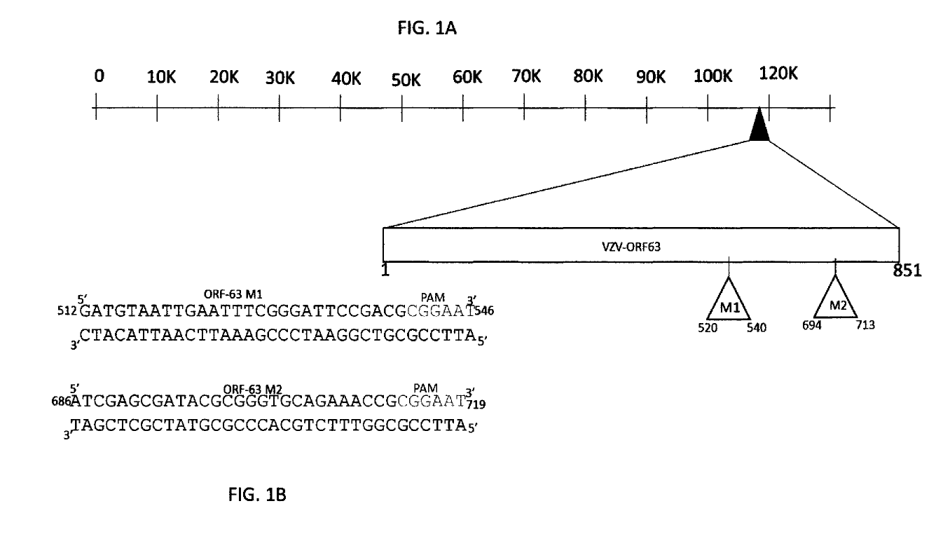

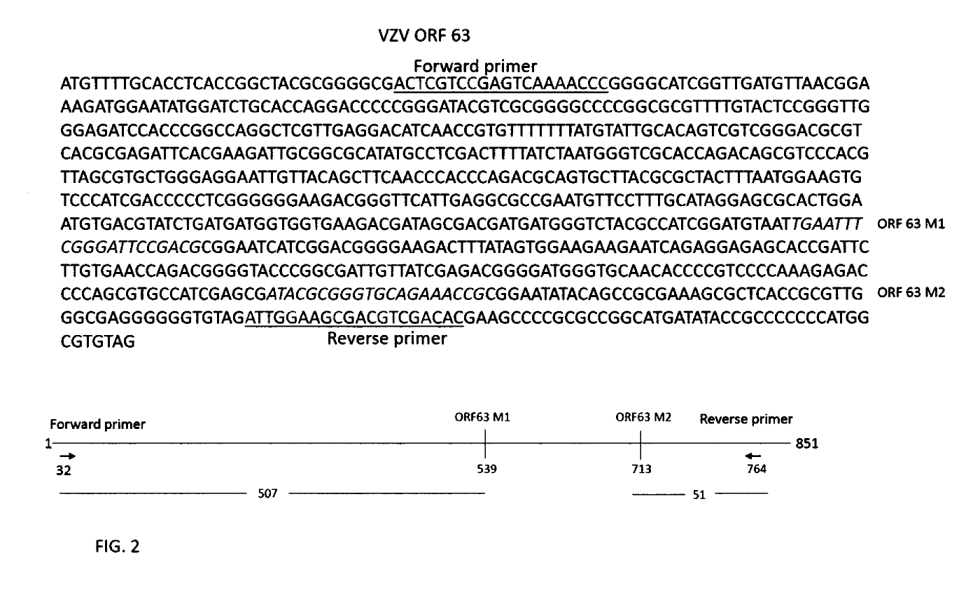

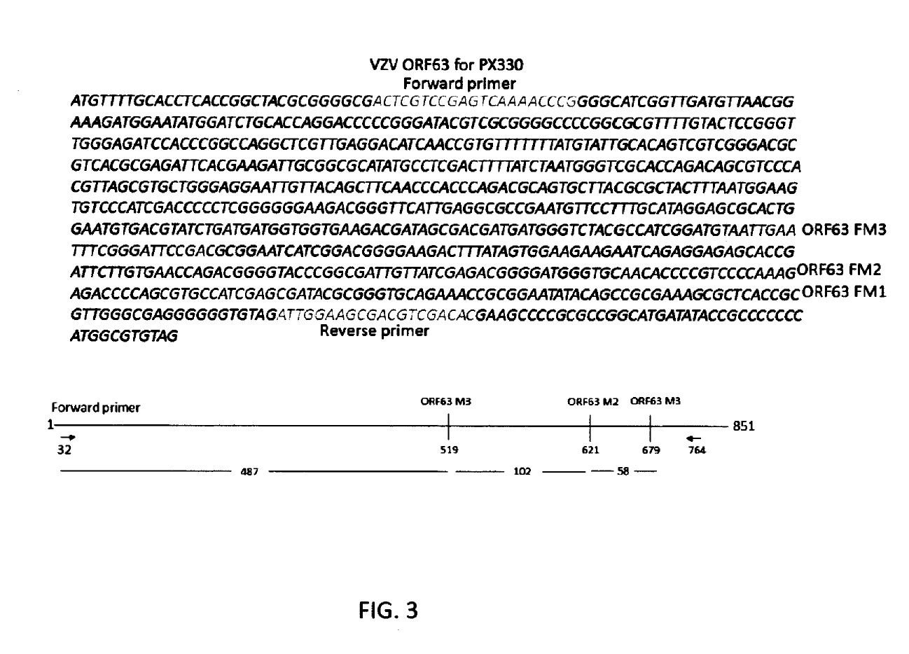

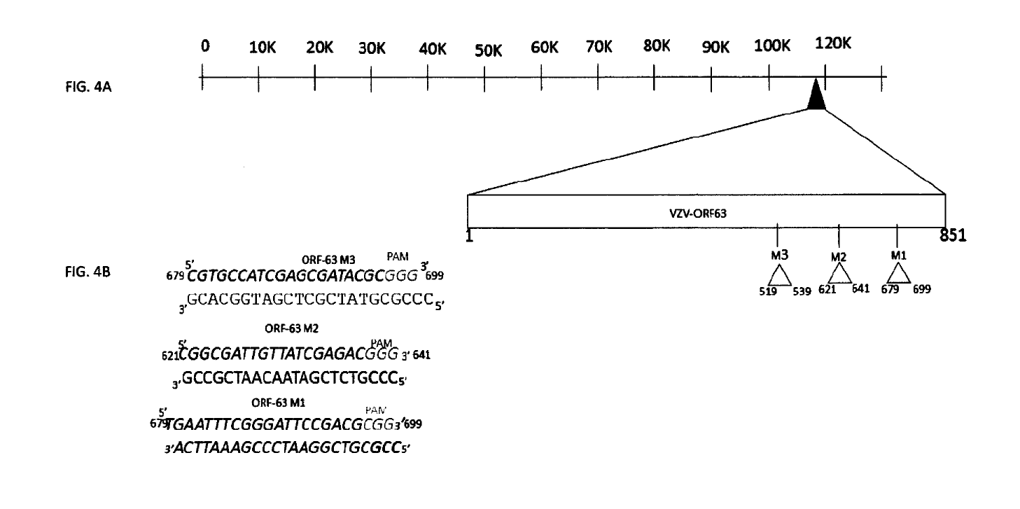

### DETAILED DESCRIPTION

Viruses, such as the Herpesviridae virus family, including Varicella Zoster virus (VZV), Epstein-Barr virus (EBV), and human papillomavirus (HPV) have the ability to lie dormant within a cell indefinitely and not be fully eradicated even after treatment. The result is that the virus can reactivate and begin producing large amounts of viral progeny without the host being infected by any new outside virus. In the latent state, the viral genome persists within the host cells as episomes; stabilized and floating in the cytoplasm or nucleus. For these latent viruses, it has not been possible to find therapeutic approaches which completely eradicate such infections.

Accordingly, embodiments of the invention are directed to compositions and methods for the treatment and eradication of latent viruses from a host cell or a subject. Methods of the invention may be used to remove viral or other foreign genetic material from a host organism, without interfering with the integrity of the host's genetic material. A nuclease may be used to target viral nucleic acid, thereby interfering with viral replication or transcription or even excising the viral genetic material from the host genome. The nuclease may be specifically targeted to remove only the viral nucleic acid without acting on host material either when the viral nucleic acid exists as a particle within the cell or when it is integrated into the host genome. Targeting the viral nucleic acid can be done using a sequence-specific moiety such as a guide RNA that targets viral genomic material for destruction by the nuclease and does not target the host cell genome. In some embodiments, a CRISPR/Cas nuclease and guide RNA (gRNA) that together target and selectively edit or destroy viral genomic material is used. The CRISPR (clustered regularly interspaced short palindromic repeats) is a naturally-occurring element of the bacterial immune system that protects bacteria from phage infection. The guide RNA localizes the CRISPR/Cas complex to a viral target sequence. Binding of the complex localizes the Cas endonuclease to the viral genomic target sequence causing breaks in the viral genome. Other nuclease systems can be used including, for example, zinc finger nucleases, transcription activator-like effector nucleases (TALENs), meganucleases, or any other system that can be used to degrade or interfere with viral nucleic acid without interfering with the regular function of the host's genetic material.

The compositions may be used to target viral nucleic acid in any form or at any stage in the viral life cycle. The targeted viral nucleic acid may be present in the host cell as independent particles. In a preferred embodiment, the viral infection is latent and the viral nucleic acid is integrated into the host genome. Any suitable viral nucleic acid may be targeted for cleavage and digestion.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

All genes, gene names, and gene products disclosed herein are intended to correspond to homologs from any species for which the compositions and methods disclosed herein are applicable. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, are intended to encompass homologous and/or orthologous genes and gene products from other species.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“About” as used herein when referring to a measurable value such as an amount, a temporal duration, and the like, is meant to encompass variations of +/−20%, +/−10%, +/−5%, +1-1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein the terms “antibiotic, antibacterial, antimycotic, antiviral, antiproliferative or antineoplastic drugs and agents” are intended to include any drug, agent or compound having an antibiotic, antibacterial, antimycotic, antiviral, antiproliferative or antineoplastic effect in an animal, preferably a human. In particular, the term “antimicrobial drug” will be understood to encompass said antibiotic, antibacterial, antimycotic, and antiviral compounds, as well as other compounds that have an antimicrobial effect (such as anti-plasmodial drugs).

For the purposes of this invention, the term “antimicrobial drug” is intended to encompass any pharmacological agent effective in inhibiting, attenuating, combating or overcoming infection of mammalian cells by a microbial pathogen in vivo or in vitro. Antimicrobial drugs as provided as components of the antimicrobial agents of the invention include but are not limited to penicillin and drugs of the penicillin family of antimicrobial drugs, including but not limited to penicillin-G, penicillin-V, phenethicillin, ampicillin, amoxacillin, cyclacillin, bacampicillin, hetacillin, cloxacillin, dicloxacillin, methicillin, nafcillin, oxacillin, azlocillin, carbenicillin, mezlocillin, piperacillin, ticaricillin, and imipenim; cephalosporin and drugs of the cephalosporin family, including but not limited to cefadroxil, cefazolin, caphalexin, cephalothin, cephapirin, cephradine, cefaclor, cefamandole, cefonicid, cefoxin, cefuroxime, ceforanide, cefotetan, cefinetazole, cefoperazone, cefotaxime, ceftizoxime, ceftizone, moxalactam, ceftazidime, and cefixime; aminoglycoside drugs and drugs of the aminoglycoside family, including but not limited to streptomycin, neomycin, kanamycin, gentamycin, tobramycin, amikacin, and netilmicin; macrolide and drugs of the macrolide family, exemplified by azithromycin, clarithromycin, roxithromycin, erythromycin, lincomycin, and clindamycin; tetracycline and drugs of the tetracycline family, for example, tetracycline, oxytetracycline, democlocyclin, methacyclin, doxycyclin, and minocyclin; quinoline and quinoline-like drugs, such as, for example, naladixic acid, cinoxacin, norfloxacin, ciprofloxacin, ofloxicin, enoxacin, and pefloxacin; antimicrobial peptides, including but not limited to polymixin B, colistin, and bacitracin, as well as other antimicrobial peptides such as defensins (Lehrer et al., 1991, Cell 64: 229-230), magainins (Zasloff, 1987, Proc. Natl. Acad. Sci. USA 84: 5449-5453), cecropins (Lee et al., 1989, Proc. Natl. Acad. Sci. USA 86: 9159-9162 and Boman et al., 1990, Ear. J. Biochem. 201: 23-31), and others, provided as naturally-occurring, chemically synthesized in vitro or produced as the result of engineering to make such peptides resistant to the action of pathogen-specific proteases and other deactivating enzymes; other antimicrobial drugs, including chloramphenicol, vancomycin, rifampicin, metronidazole, ethambutol, pyrazinamide, sulfonamides, isoniazid, and erythromycin.

Antiviral drugs, including but not limited to reverse transcriptase inhibitors, protease inhibitors, antiherpetics such as acyclovir and gancyclovir, azidothymidine, cytidine arabinoside, ribavirin, amantadine, iododeoxyuridine, foscamet, trifluoridine, methizazone, vidarabine and levanisole are also encompassed by this definition and are expressly included therein. Antimycotic drugs provided by the invention and comprising the pharmaceutical compositions thereof include but are not limited to clotrimazole, nystatin, econazole and myconixole, ketoconazole, grisefulvin, ciclopixox, naftitine and other imidizole antimycotics. Antiproliferative and antineoplastic agents provided by the invention and comprising the pharmaceutical compositions thereof include but are not limited to methotrexate, doxarubicin, daunarubicin, epipodophyllotoxins, 5-fluorouracil, tamoxifen, actinomycin D, vinblastine, vincristine, colchicine and taxol.

The term “eradication” of virus, e.g. VZV, as used herein, means that that virus is unable to replicate, the genome is deleted, fragmented, degraded, genetically inactivated, or any other physical, biological, chemical or structural manifestation, that prevents the virus from being transmissible or infecting any other cell or subject resulting in the clearance of the virus in vivo. In some cases, fragments of the viral genome may be detectable, however, the virus is incapable of replication, or infection etc.

An “effective amount” as used herein, means an amount which provides a therapeutic or prophylactic benefit. “Effective amount” in the context of the invention refers to an amount of a composition or isolated nucleic acid sequence embodied herein, that expresses at least one gRNA and a nuclease, e.g. Cas9, to cut host DNA at a location(s) specified by the gRNA, which amount is obtained with the stable expression of nuclease.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Gene essential to viral replication” or “Gene essential to herpesvirus replication” refers to a gene present in a virus, e.g., a herpesvirus, the expression of which alone or in association with another viral gene is required for the virus to replicate and maintain its normal life cycle. Herpesviruses have been well studied, in particular those that infect humans, and there are a number of genes in each of Herpes simplex viruses 1 and 2, varicella zoster virus, EBV (Epstein Barr virus), human cytomegalovirus, human herpesvirus 6, human herpesvirus 7, and Kaposi's sarcoma associated herpesvirus (human herpesvirus 8) which have been identified to be essential to virus replication.

“Herpesviridae” or herpesviruses refers to a large family of DNA viruses that cause diseases in animals, including humans. The members of this family are also known as herpesviruses. The family name is derived from the Greek word herpein (“to creep”), referring to the latent, recurring infections typical of this group of viruses. Herpesviridae can cause latent or lytic infections. There are more than 130 herpesviruses, and some are from mammals, birds, fish, reptiles, amphibians, and mollusks. Of these there are eight known herpesvirus types: Herpes simplex viruses 1 and 2, varicella zoster virus, EBV (Epstein Barr virus), human cytomegalovirus, human herpesvirus 6, human herpesvirus 7, and Kaposi's sarcoma associated herpesvirus. Of these eight, there are at least five species of Herpesviridae which are extremely widespread among humans, HSV 1, which causes facial/oral cold sores, HSV 2 (genital herpes), Varicella zoster virus, which causes chicken pox and shingles, Epstein Barr virus, which causes mononucleosis (glandular fever) and Cytomegalovirus which are extremely widespread among humans. More than 90% of adults have been infected with at least one of these, and a latent form of the virus remains in most people.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

The term “target nucleic acid” sequence refers to a nucleic acid (often derived from a biological sample), to which the oligonucleotide is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The term “polynucleotide” is a chain of nucleotides, also known as a “nucleic acid”. As used herein polynucleotides include, but are not limited to, all nucleic acid sequences which are obtained by any means available in the art, and include both naturally occurring and synthetic nucleic acids.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “percent sequence identity” or having “a sequence identity” refers to the degree of identity between any given query sequence and a subject sequence.

The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

Where any amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

Compositions for Eradication of Virus

Compositions for eradication of a herpesvirus, e.g. Varicella zoster virus (VZV) include using a targeted nuclease which specifically targets viral nucleic acid sequences for destruction and eradication of that virus in a host cell in vitro or in vivo. Any suitable nuclease systems can be used including, for example, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof. See Schiffer, 2012, J Virol 88(17):8920-8936, incorporated by reference. In preferred embodiments, the system is a clustered regularly interspaced short palindromic repeat (CRISPR) nuclease system.

CRISPR (Clustered Regularly Interspaced Short Palindromic Repeats) is found in bacteria and is believed to protect the bacteria from phage infection. It has recently been used as a means to alter gene expression in eukaryotic DNA, but has not been proposed as an anti-viral therapy or more broadly as a way to disrupt genomic material. Rather, it has been used to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327; Bhaya et al., Annu Rev Genet (2011) 45:273-297; Wiedenheft et al., Nature (2012) 482:331-338); Jinek M et al., Science (2012) 337:816-821; Cong L et al., Science (2013) 339:819-823; Jinek M et al., (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918).

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell Res. 23:465-472; Hwang et al., 2013, Nat. Biotechnol. 31:227-229; Xiao et al., 2013, Nucl. Acids Res. 1-11.

Three types (I-III) of CRISPR systems have been identified. CRISPR clusters contain spacers, the sequences complementary to antecedent mobile elements. CRISPR clusters are transcribed and processed into mature CRISPR RNA (crRNA). In embodiments, the CRISPR/Cas system can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In type II CRISPR systems, correct processing of pre crRNA requires a trans encoded small RNA (tracrRNA), endogenous nuclease 3 (rnc) and a Cas9 protein. The tracrRNA serves as a guide for nuclease 3 aided processing of pre crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′ 5′ exonucleolytically. In nature, DNA binding and cleavage typically requires protein and both RNA species. However, guide RNAs can be engineered so as to incorporate aspects of both the crRNA and tracrRNA into a single RNA molecule. (See, e.g., Jinek M., et. al. 2012 Science 337:816 821 the entire contents of which is hereby incorporated by reference). The tracrRNA and spacer RNA together are often referred to as guide RNA, which is typically between 17 and 20 nucleotides in length. The two RNA species can be joined to form one hybrid RNA molecule referred to herein as “guide RNA” (gRNA). When complexed with CAS9, the CAS9 guide RNA complex will find and specifically cut the correct DNA targets. (Pennisi, E. 2013 Science 341 (6148): 833 836). Thus, reference herein to a gRNA “targeted to” a component, including a specific protein, of a viral genome refers to a CRISPR Cas system gRNA that hybridizes with the specified target sequence, whereby the gRNA hybridizes to the targeted sequence and the CRISPR associated Cas9 nuclease cleaves the targeted viral DNA molecule.

In certain embodiments, the CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNase domains, protein-protein interaction domains, dimerization domains, as well as other domains.

In one embodiment, the RNA-guided endonuclease is derived from a type II CRISPR/Cas system. The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3^{rd} nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately.

In other embodiments. the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

The CRISPR-associated endonuclease Cas9 nuclease can have a nucleotide sequence identical to the wild type Streptococcus pyogenes sequence. The CRISPR-associated endonuclease may be a sequence from other species, for example other Streptococcus species, such as thermophiles. The Cas9 nuclease sequence can be derived from other species including, but not limited to: Nocardiopsis dassonvillei, Streptomyces pristinaespiralis, Streptomyces viridochromogenes, Streptomyces roseurn, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficle, Finegoldia magna, Natranaerobius thermophiles, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. Pseudomonas aeruginosa, Escherichia coli, or other sequenced bacteria genomes and archaea, or other prokaryotic microorganisms may also be a source of the Cas9 sequence utilized in the embodiments disclosed herein.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

The wild type Streptococcus pyogenes Cas9 sequence can be modified. The nucleic acid sequence can be codon optimized for efficient expression in mammalian cells, i.e., “humanized.” sequence can be for example, the Cas9 nuclease sequence encoded by any of the expression vectors listed in Genbank accession numbers KM099231.1 GI:669193757; KM099232.1 GI:669193761; or KM099233.1 GI:669193765. Alternatively, the Cas9 nuclease sequence can be for example, the sequence contained within a commercially available vector such as PX330 or PX260 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GI:669193757; KM099232.1 GI:669193761; or KM099233.1 GI:669193765 or Cas9 amino acid sequence of PX330 or PX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type Cas9 polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the Cas9 amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site currently maintained by the California Institute of Technology displays structures of non-natural amino acids that have been successfully incorporated into functional proteins).

Guide RNA sequences according to the present invention can be sense or anti-sense sequences. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophiles requires 5′-NNAGAA for CRISPR 1 and 5′-NGGNG for CRISPR3 and Neiseria meningitidis requires 5′-NNNNGATT. The specific sequence of the guide RNA may vary, but, regardless of the sequence, useful guide RNA sequences will be those that minimize off-target effects while achieving high efficiency and complete ablation of the herpesvirus, for example, VZV. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs. Accordingly, in some embodiments, a polynucleotide sequence encoding at least one gRNA may encode two distinct gRNA sequences. In other embodiments, one polynucleotide encodes for one gRNA; a second polynucleotide encodes for a second gRNA; a third polynucleotide encodes for a third gRNA, etc., wherein each gRNA is complementary to distinct sequences of a target nucleic acid sequence. In other embodiments, a polynucleotide sequence encodes for two or more distinct gRNA sequences. In other embodiments, a polynucleotide encodes multiple gRNA sequences having overlapping target nucleic acid sequences. The combinations of gRNAs encoded by the polynucleotides is limited only by the imagination of the user.

A CRISPR/Cas9 gene editing complex of the invention works optimally with a guide RNA that targets the viral genome. Guide RNA (gRNA) (which includes single guide RNA (sgRNA), crisprRNA (crRNA), transactivating RNA (tracrRNA), any other targeting oligonucleotide, or any combination thereof) leads the CRISPR/Cas9 complex to the viral genome in order to cause viral genomic disruption. In an aspect of the invention, CRISPR/Cas9/gRNA complexes are designed to target Herpesviridae, e.g. VZV, within a cell. It should be appreciated that any virus can be targeted using the composition of the invention. Identification of specific regions of the virus genome aids in development and designing of CRISPR/Cas9/gRNA complexes. In an aspect of the invention, the CRISPR/Cas9/gRNA complexes are designed to target latent viruses within a cell. Once transfected within a cell, the CRISPR/Cas9/gRNA complexes cause repeated insertions or deletions to render the viral genome incapacitated, or due to number of insertions or deletions, the probability of repair is significantly reduced.

The compositions and methods of the present invention may include a sequence encoding a guide RNA that is complementary to a target sequence in a herpesvirus, for example, VZV. Varicella-zoster virus (VZV) is an alpha-herpesvirus that is in the same subfamily as herpes simplex virus (HSV) 1 and 2. VZV is a member of varicellovirus genus, along with equine herpesvirus 1 and 4, pseudorabies virus, and bovine herpesvirus 1 and 5. Ceropithecine herpesvirus 9 (simian varicella virus) is virus most homologous to VZV.

In some embodiments, the herpesvirus comprises: herpes simplex virus (HSV)-1, HSV-2, varicella zoster virus (VZV), human herpesvirus (HHV)-5 HHV-6, HHV-7, cytomegalovirus, Epstein Barr Virus, herpes zoster virus (HZ), equine herpesvirus 1 and 4, pseudorabies virus, bovine herpesvirus 1 and 5, HHV6A and HHV6B or herpes lymphotropic virus, HHV7 or Pityriasis Rosacea, SHV/HHV8 or simian varicella virus (herpes virus 9).

VZV genome: The complete sequence of the VZV genome was determined by Davison and Scott (J Gen Virol. 1986; 67:1759-1816.). The prototype strain, VZV Dumas is 124,884 base pairs in length. The genome consists of a unique long region of ˜105,000 bp, (UL) bounded by terminal long (TRL) and internal long (IRL) repeats, and a unique short region of ˜5,232 bp (US) bounded by internal short (IRS), and terminal short (TRS) repeats. The US region can orientate either of two directions, while the UL region rarely changes its orientation; thus, there are usually two isomers of the genome in infected cells.

The VZV genome is linear in virions with an unpaired nucleotide at each end. In VZV-infected cells the ends pair and the genome circularizes. The genome has five repeat regions. Repeat region 1 (R1) is located in open reading frame (ORF) 11, R2 is located in ORF14 (glycoprotein C), R3 in ORF22, R4 between ORF62 and the origin of viral replication, and R5 between ORF 60 and 61. The length of the repeat regions varies among different VZV strains and has been used to distinguish the strains. The genes that encode ORF62 and ORF70, ORF63 and ORF71, and ORF64 and ORF69 are duplicated. The origin of replication (ori) is located in the repeat region. About two-thirds of VZV ORFS are necessary for replication in vitro, most of which are among the ˜40 genes that are conserved in all herpesviruses, including eight glycoproteins (gB, gC, gE, gH, gI, gK, gL, gN), proteins that are involved in DNA replication and other functions, such as DNA cleavage and packaging, nucleic acid metabolism and capsid assembly. Replication proteins include the small and large subunits of the viral ribonucleotide reductase (known as ORF18 and ORF19), the two subunits of the viral DNA polymerase (known as ORF16 and ORF28), the single-stranded DNA-binding protein (known as ORF29), the origin of DNA replication binding protein (known as ORF51), two viral protein kinases (known as ORF47 and ORF66) and other enzymes that are involved in DNA replication, including dUTPase (known as ORF 8), thymidylate synthetase (known as ORF13), DNase (known as ORF48) and uracil DNA glycosylase (known as ORF59). Some VZV gene products have functional subdomains that are dispensable in cultured cells; others are dispensable for replication in vitro but are necessary for pathogenesis. The ORF9-ORF12 cluster of tegument proteins is conserved in the alpha-herpesviruses. The products of the dispensable genes are of interest for their potential differential functions in tropism. Cloning the VZV genome into bacterial artificial chromosome vectors or as four or five overlapping fragments in cosmids enables the deletion of ORFs or targeted mutations of coding and non-coding sequences to define functions in vitro and in vivo.

VZV immediate-early genes: VZV encodes at least 70 genes, three (ORF62, 63, 64) are which are present in both of the short repeat regions (Cohen et al. Varicella-zoster virus: Replication, pathogenesis, and management. In: Knipe, D M.; Howley, P M., editors. Fields Virology. 5^{th} ed. Philadelphia: Lippincott-Williams & Wilkins; 2007b). VZV encodes at least 3 immediate-early (IE) proteins that are located in the tegument of virions and regulate virus transcription. 1E4 and IE62 transactivate IE, late, and early promoters. IE63 represses several VZV promoters, and inhibits the activity of interferon-alpha (Ambagala et al., J Virol. 2007; 81:7844-7851), and binds to anti-silencing protein 1 (Ambagala et al., J Viral. 2009; 83:200-209). ORF61 protein, which is not present in the tegument of virions and has not been shown to be an IE gene, activates IE, early, and late viral promoters.

VZV genes encoding replication proteins: VZV encodes a viral DNA polymerase, likely composed of two subunits (ORF28 and ORF16) that is inhibited by acyclovir. The viral thymidine kinase (ORF36) phosphorylates deoxycytidine, thymidine, and acyclovir. VZV ORF18 and ORF19 encode the small and large subunits of ribonucleotide reductase which convert ribonucleotides to deoxyribonucleotides. VZV encodes at least two DNA binding proteins-ORF29 protein is a single-stranded DNA binding protein, and ORF 51 protein binds to the origin of DNA replication. VZV encodes two protein kinases. ORF47 protein phosphorylates VZV ORF32 protein, IE62, IE63, and glycoprotein I. ORF66 protein phosphorylates IE62 which results inclusion of IE62 into the virion tegument. VZV encodes other enzymes including a dUTPase (ORFS), thymidylate synthetase (ORF13), protease (ORF33), DNase (ORF48), and uracil DNA glycosylase (ORF59).

VZV genes encoding putative late proteins: VZV ORF10 encodes a tegument protein that forms a complex with transcription factors at the ORF62 promoter to activate transcription of ORF62. ORF17 protein induces cleavage of RNA. ORF33.5 encodes the assembly protein which forms a scaffold thought be involved in construction of nucleocapsids. ORF40 encodes the major nucleocapsid protein, while ORF21 also encodes a nucleocapsid protein. ORF54 encodes the putative portal protein which allows viral DNA to enter nucleocapsids.

VZV genes encoding glycoproteins: VZV encodes 7 viral glycoproteins-gB (ORF31), gC (ORF14), gE (OEF68), gH (ORF 37), gI (ORF67), gK (ORF 5), gL (ORF 60), gM (ORF50), and presumably gN (ORF9A). VZV gB, based on homology with HSV gB, is likely critical for entry of virus into cells. gE is binds to a cellular receptor (insulin degrading enzyme [Li et al., Cell. 2006; 127:305-316) and gH and gM are important for cell-to-cell spread of virus (Yamagishi et al., J Virol. 2008; 82:795-804). gI facilitates maturation of gE, and gL is a chaperone for gH. gK may be important for syncytia formation.

Core proteins conserved with Herpesviridae in other subfamilies: The VZV genome contains about 41 “core genes” that are conserved with each of the three subfamilies of herpesviruses, alpha-herpesvirus, beta-herpesvirus, and gamma-herpesvirus (Davison A. J., Rev Med Virol. 1993; 3:237-244). Core genes include 1E4, the VZV DNA polymerase, helicase-primase components, single-stranded DNA-binding protein, ribonucleotide reductase, uracil-DNA glycosylase, dUTPase, DNase, ORF47 protein kinase, major capsid protein, protease, assembly protein, several tegument proteins, gB, gH, gL, gM, and gN.

In certain embodiments, a composition for eradicating a VZV in vitro or in vivo, comprises an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a VZV genome.

In another embodiment, a target nucleic acid sequence comprises one or more nucleic acid sequences having at least a 75% sequence identity to coding and non-coding nucleic acid sequences of the VZV genome. In another embodiment, a target nucleic acid sequence comprises one or more nucleic acid sequences in coding and non-coding nucleic acid sequences of the VZV genome.

In some embodiments, the polynucleotide sequence encoding at least one gRNA may encode a gRNA targeted to a varicella zoster virus (VZV) or a simian varicella virus (SVV) protein comprising VZV glycoprotein E, VZV viral kinase ORF47, VZV viral kinase ORF66, VZV 1E62 protein, VZV IE63 protein, VZV IE70 protein, VZV IE71 protein, VZV DNA polymerase, and a VZV glycoprotein, ORF 63/70, ORF 62/71, ORF6, ORF28, ORF55, ORF25, ORF26, ORF30, ORF34, ORF 42/45, ORF 43, ORF54, ORF4, ORF5, ORF9A, ORF9, ORF 17, ORF20, ORF21, ORF22, ORF24, ORF27, ORF29, ORF 31, ORF33, ORF33.5, ORF37, ORF38, ORF39, ORF40, ORF41, ORF44, ORF46, ORF48, ORF50, ORF51, ORF52, ORF53, ORF56, ORF60, ORF61, ORF62, ORF64, ORF65, ORF66, ORF67, ORF68, and/or ORF69.

In certain embodiments, a target nucleic acid sequence has at least a 75% sequence identity to nucleic acid sequences in unique long region (UL), terminal long (TRL) and internal long (IRL) repeats, unique short region (US), internal short repeats (IRS), terminal short repeats (TRS), open reading frames (ORF), glycoproteins, isomers or combinations thereof. In certain embodiments, a gRNA sequence comprises a target nucleic acid sequence in unique long region (UL), terminal long (TRL) and internal long (IRL) repeats, unique short region (US), internal short repeats (IRS), terminal short repeats (TRS), open reading frames (ORF), glycoproteins, isomers or combinations thereof.

In certain embodiments, the polynucleotide sequence encoding at least one gRNA may encode a gRNA targeted to a Herpes simplex virus type 1 (HSV 1) protein comprising DNA Polymerase (UL42), DNA Polymerase Catalytic Subunit (UL30), DNA Helicase (UL5), DNA Primase (UL52), ICP4 (transcriptional regulator), US 1 (host range factor), UL49A (envelope protein), ICPO (transcriptional regulator), UL1, UL8, UL9, UL 14, UL15, UL17, UL18, UL19, UL22, UL25, U126, UL26.5, UL27, UL28, UL29 UL31, LTL34, UL35, UL36, UL37, UL38, UL48, UL49, UL49.5, UL53, UL54, RS I, and/or US6.

In certain embodiments, the polynucleotide sequence encoding at least one gRNA may encode a gRNA targeted to a Herpes simplex virus type 2 (HSV 2) protein comprising DNA Polymerase (UL42), DNA Polymerase Catalytic Subunit (UL30), DNA Helicase (UL5), DNA Primase (UL52), ICP4 (transcriptional regulator), US1 (host range factor), UL49A (envelope protein), ICPO (transcriptional regulator), UL 1, UL8, UL9, UL14, UL15, UL17, UL18, UL19, UL22, UL25, U126, UL26.5, UL27, UL28, UL29 UL31, UL34, UL35, UL36, UL37, UL38, UL48, UL49, UL49.5, UL53, UL54, RS I, and/or US6.

In other embodiments, the target nucleic acid sequence has at least a 75% sequence identity to one or more nucleic acid sequences encoding immediate early gene products, replication proteins, putative late proteins, glycoproteins, or combinations thereof. In other embodiments, the target nucleic acid sequence comprises one or more nucleic acid sequences encoding immediate early gene products, replication proteins, putative late proteins, glycoproteins, or combinations thereof.

In other embodiments the target nucleic acid sequences comprise nucleic acid sequences having at least a 75% sequence identity to one or more open reading frame (ORE) sequences. In other embodiments the target nucleic acid sequences comprise nucleic acid sequences in one or more open reading frame (ORF) sequences.

Non-limiting examples of nucleic acid sequences comprising gRNA nucleic acid sequences are as follows:

(Sacas9 ORF63 FM1; SEQ ID NO: 1)
5′CACCGtgaatttcgggattccgacg-3′;
(Sacas9 ORF63 RM1; SEQ ID NO: 2)
5′-AAACcgtcggaatcccgaaattcaC-3′;
(Sacas9 ORF63 FM2; SEQ ID NO: 3)
5′CACCGatacgcgggtgcagaaaccg-3′;
(Sacas9 ORF63 RM2; SEQ ID NO: 4)
5′-AAACcggtttctgcacccgcgtatC-3′;
(wtCas9 ORF63 FM3; SEQ ID NO: 5)
5′-CGTGCCATCGAGCGATACGCGGG-3′;
(wtCas9 ORF63 RM3; SEQ ID NO: 6)
5′-CCCGCGTATCGCTCGATGGCACG-3′;
(wtCas9 ORF63 FM2; SEQ ID NO: 7)
5′-CGGCGATTGTTATCGAGACGGG-3′;
(wtCas9 ORF63 RM2; SEQ ID NO: 8)
5′CCCGTCTCGATAACAATCGCCG-3′;
(wtCas9 ORF63 FM1; SEQ ID NO: 9)
5′-TGAATTTCGGGATTCCGACGCGG-3′;
(wtCas9 ORF63 RM1; SEQ ID NO: 10)
5′-CCGCGTCGGAATCCCGAAATTAC-3′.

In other embodiments, nucleic acid sequences comprising the gRNA sequences have at least a 75% sequence identity to sequences comprising: SEQ ID NOS: 1-10, or combinations thereof. In other embodiments, nucleic acid sequences comprising the gRNA sequences comprise: SEQ ID NOS: 1-10, or combinations thereof.

In certain embodiments, an isolated nucleic acid sequence comprises a nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a VZV genome. In other embodiments, the isolated nucleic acid sequences further comprise a short proto-spacer adjacent motif (PAM)-presenting DNA oligonucleotide sequence.

When the compositions are administered as a nucleic acid or are contained within an expression vector, the CRISPR endonuclease can be encoded by the same nucleic acid or vector as the guide RNA sequences. Alternatively, or in addition, the CRISPR endonuclease can be encoded in a physically separate nucleic acid from the gRNA sequences or in a separate vector.

In some embodiments, specific CRISPR/Cas/gRNA complexes are introduced into a cell. A guide RNA is designed to target at least one category of sequences of the viral genome. In addition to latent infections this invention can also be used to control actively replicating viruses by targeting the viral genome before it is packaged or after it is ejected.

In some embodiments, a cocktail of guide RNAs may be introduced into a cell. The guide RNAs are designed to target numerous categories of sequences of the viral genome. By targeting several areas along the genome, the double strand break at multiple locations fragments the genome, lowering the possibility of repair. Even with repair mechanisms, the large deletions render the virus incapacitated.

In some embodiments, several guide RNAs are added to create a cocktail to target different categories of sequences. For example, two, five, seven or eleven guide RNAs may be present in a CRISPR cocktail targeting three different categories of sequences. However, any number of gRNAs may be introduced into a cocktail to target categories of sequences. In preferred embodiments, the categories of sequences are important for genome structure, host cell transformation, and infection latency, respectively.

In some aspects of the invention, in vitro experiments allow for the determination of the most essential targets within a viral genome. For example, to understand the most essential targets for effective incapacitation of a genome, subsets of guide RNAs are transfected into model cells. Assays can determine which guide RNAs or which cocktail is the most effective at targeting essential categories of sequences.

Modified or Mutated Nucleic Acid Sequences: In some embodiments, any of the nucleic acid sequences may be modified or derived from a native nucleic acid sequence, for example, by introduction of mutations, deletions, substitutions, modification of nucleobases, backbones and the like. The nucleic acid sequences include the vectors, gene-editing agents, gRNAs, tracrRNA etc. Examples of some modified nucleic acid sequences envisioned for this invention include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, modified oligonucleotides comprise those with phosphorothioate backbones and those with heteroatom backbones, CH₂—NH—O—CH₂, CH, —N(CH₃)—O—CH₂ [known as a methylene(methylimino) or MMI backbone], CH₂—O—N(CH₃)—CH₂, CH₂—N (CH₃)—N(CH₃)—CH, and O—N(CH₃)—CH₂—CH₂ backbones, wherein the native phosphodiester backbone is represented as O—P—O—CH,). The amide backbones disclosed by De Mesmaeker et al. Acc. Chem. Res. 1995, 28:366-374) are also embodied herein. In some embodiments, the nucleic acid sequences having morpholino backbone structures (Summerton and Weller, U.S. Pat. No. 5,034,506), peptide nucleic acid (PNA) backbone wherein the phosphodiester backbone of the oligonucleotide is replaced with a polyamide backbone, the nucleobases being bound directly or indirectly to the aza nitrogen atoms of the polyamide backbone (Nielsen et al. Science 1991, 254, 1497). The nucleic acid sequences may also comprise one or more substituted sugar moieties. The nucleic acid sequences may also have sugar mimetics such as cyclobutyls in place of the pentofuranosyl group.

The nucleic acid sequences may also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalldyamino)adenine or other heterosubstituted alkyladenines, 2-thiouracil, 2-thiothymine, 5-bromouracil, 5-hydroxymethyluracil, 8-azaguanine, 7-deazaguanine, N₆ (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine may be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278).

Another modification of the nucleic acid sequences of the invention involves chemically linking to the nucleic acid sequences one or more moieties or conjugates which enhance the activity or cellular uptake of the oligonucleotide. Such moieties include but are not limited to lipid moieties such as a cholesterol moiety, a cholesteryl moiety (Letsinger et al., Proc. Natl. Acad. Sci. USA 1989, 86, 6553), cholic acid (Manoharan et al. Bioorg. Med. Chem. Let. 1994, 4, 1053), a thioether, e.g., hexyl-S-tritylthiol (Manoharan et al. Ann. N.Y. Acad. Sci. 1992, 660, 306; Manoharan et al. Bioorg. Med. Chem. Let. 1993, 3, 2765), a thiocholesterol (Oberhauser et al., Nucl. Acids Res. 1992, 20, 533), an aliphatic chain, e.g., dodecandiol or undecyl residues (Saison-Behmoaras et al. EMBO J. 1991, 10, 111; Kabanov et al. FEBS Lett. 1990, 259, 327; Svinarchuk et al. Biochimie 1993, 75, 49), a phospholipid, e.g., di-hexadecyl-rac-glycerol or triethylammonium 1,2-di-O-hexadecyl-rac-glycero-3-H-phosphonate (Manoharan et al. Tetrahedron Lett. 1995, 36, 3651; Shea et al. Nucl. Acids Res. 1990, 18, 3777), a polyamine or a polyethylene glycol chain (Manoharan et al. Nucleosides & Nucleotides 1995, 14, 969), or adamantane acetic acid (Manoharan et al. Tetrahedron Lett. 1995, 36, 3651).

It is not necessary for all positions in a given nucleic acid sequence to be uniformly modified, and in fact more than one of the aforementioned modifications may be incorporated in a single nucleic acid sequence or even at within a single nucleoside within a nucleic acid sequence.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-dimethylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶Nmethyladenosine; N⁶, N⁶-dimethyladenosine; N⁶,2′-O-trimethyladenosine; 2 methylthio-N⁶Nisopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶ threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1;2′-O-dimethyl inosine; 2′-O-methyl guanosine; 1-methyl guanosine; N²-methyl guanosine; N², N²-dimethyl guanosine; N², 2′-O-dimethyl guanosine; N², N², 2′-O-trimethyl guanosine; 2′-O-ribosyl guanosine (phosphate); 7-methyl guanosine; N²;7-dimethyl guanosine; N²; N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

The isolated nucleic acid molecules of the present invention can be produced by standard techniques. For example, polymerase chain reaction (PCR) techniques can be used to obtain an isolated nucleic acid containing a nucleotide sequence described herein. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Delivery Vehicles

Delivery vehicles as used herein, include any types of molecules for delivery of the compositions embodied herein, both for in vitro or in vivo delivery. Examples, include, without limitation: expression vectors, nanoparticles, colloidal compositions, lipids, liposomes, nanosomes, carbohydrates, organic or inorganic compositions and the like.

Any suitable method can be used to deliver the compositions to the infected cell or tissue. For example, the nuclease or the gene encoding the nuclease may be delivered by injection, orally, or by hydrodynamic delivery. The nuclease or the gene encoding the nuclease may be delivered to systematic circulation or may be delivered or otherwise localized to a specific tissue type. The nuclease or gene encoding the nuclease may be modified or programmed to be active under only certain conditions such as by using a tissue-specific promoter so that the encoded nuclease is preferentially or only transcribed in certain tissue types.

In some embodiments, a delivery vehicle is an expression vector, wherein the expression vector comprises an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a VZV genome. In certain embodiments, the nuclease is a Cas9 endonuclease and a guide RNA that specifically targets a portion of a VZV genome. The Cas9 endonuclease and the guide RNA may be co-expressed in a host cell infected by a virus.

Nucleic acids as described herein may be contained in vectors. Vectors can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FLAG™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of SV40 and known bacterial plasmids, e.g., E. coli plasmids col El, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences.

Several delivery methods may be utilized in conjunction with the isolated nucleic acid sequences for in vitro (cell cultures) and in vivo (animals and patients) systems. In one embodiment, a lentiviral gene delivery system may be utilized. Such a system offers stable, long term presence of the gene in dividing and non-dividing cells with broad tropism and the capacity for large DNA inserts. (Dull et al, J Virol, 72:8463-8471 1998). In an embodiment, adeno-associated virus (AAV) may be utilized as a delivery method. AAV is a non-pathogenic, single-stranded DNA virus that has been actively employed in recent years for delivering therapeutic gene in in vitro and in vivo systems (Choi et al, Curr Gene Ther, 5:299-310, 2005). An example non-viral delivery method may utilize nanoparticle technology. This platform has demonstrated utility as a pharmaceutical in vivo. Nanotechnology has improved transcytosis of drugs across tight epithelial and endothelial barriers. It offers targeted delivery of its payload to cells and tissues in a specific manner (Allen and Cullis, Science, 303:1818-1822, 1998).

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

In some embodiments, the isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a VZV genome is operably linked to regulatory sequences (e.g., promoter, enhancer, silencer sequence, etc.) so as to retain proper transcriptional regulation.

The term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses Ad, AAV, lentivirus, and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available. A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Additional vectors include viral vectors, fusion proteins and chemical conjugates. Retroviral vectors include Moloney murine leukemia viruses and HIV-based viruses. One HIV based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector [Geller, A. I. et al., J. Neurochein, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.: 90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

In some embodiments of the invention, lentiviruses, which are a subclass of retroviruses, are used as viral vectors. Lentiviruses can be adapted as delivery vehicles (vectors) given their ability to integrate into the genome of non-dividing cells, which is the unique feature of lentiviruses as other retroviruses can infect only dividing cells. The viral genome in the form of RNA is reverse-transcribed when the virus enters the cell to produce DNA, which is then inserted into the genome at a random position by the viral integrate enzyme. The vector, now called a provirus, remains in the genome and is passed on to the progeny of the cell when it divides.

As opposed to lentiviruses, adenoviral DNA does not integrate into the genome and is not replicated during cell division. Adenovirus and the related AAV would be potential approaches as delivery vectors since they do not integrate into the host's genome. AAV can infect both dividing and non-dividing cells and may incorporate its genome into that of the host cell. For example, because of its potential use as a gene therapy vector, researchers have created an altered AAV called self-complementary adeno-associated virus (scAAV). Whereas AAV packages a single strand of DNA and requires the process of second-strand synthesis, scAAV packages both strands which anneal together to form double stranded DNA. By skipping second strand synthesis scAAV allows for rapid expression in the cell. Otherwise, scAAV carries many characteristics of its AAV counterpart. Methods of the invention may incorporate herpesvirus, poxvirus, alphavirus, or vaccinia virus as a means of delivery vectors.

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the expressed products intracellularly. See for example, Chen et al, BioTechniques, 34: 167-171 (2003), which is incorporated herein, by reference, in its entirety. The polynucleotides disclosed herein may be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Feigner and Holm, Bethesda Res. Lab. Focus, 11(2):21 (1989) and Maurer, R. A., Bethesda Res. Lab. Focus, 11(2):25 (1989).

In certain embodiments of the invention, non-viral vectors may be used to effectuate transfection. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat. No. 6,890,554 to Jesse, the contents of each of which are incorporated by reference. Delivery can be to cells (e.g. in vitro or ex vivo administration) or target tissues (e.g. in vivo administration).

Synthetic vectors are typically based on cationic lipids or polymers which can complex with negatively charged nucleic acids to form particles with a diameter in the order of 100 nm. The complex protects nucleic acid from degradation by nuclease. Moreover, cellular and local delivery strategies have to deal with the need for internalization, release, and distribution in the proper subcellular compartment. Systemic delivery strategies encounter additional hurdles, for example, strong interaction of cationic delivery vehicles with blood components, uptake by the reticuloendothelial system, kidney filtration, toxicity and targeting ability of the carriers to the cells of interest. Modifying the surfaces of the cationic non-virals can minimize their interaction with blood components, reduce reticuloendothelial system uptake, decrease their toxicity and increase their binding affinity with the target cells. Binding of plasma proteins (also termed opsonization) is the primary mechanism for RES to recognize the circulating nanoparticles. For example, macrophages, such as the Kupffer cells in the liver, recognize the opsonized nanoparticles via the scavenger receptor.

The nucleic acid sequences of the invention can be delivered to an appropriate cell of a subject. This can be achieved by, for example, the use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the nucleic acid is using liposomes, prepared by standard methods. The nucleic acids can be incorporated alone into these delivery vehicles or co-incorporated with tissue-specific antibodies, for example antibodies that target cell types that are commonly latently infected reservoirs of VZV infections. Alternatively, one can prepare a molecular complex composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding an isolated nucleic acid sequence comprising a sequence encoding a CRISPR-associated endonuclease and a guide RNA complementary to a target sequence of a VZV, as described above.

In some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol modified (PEGylated) low molecular weight LPEI. In some embodiments, the compositions can be formulated as a nanoparticle encapsulating the compositions embodied herein. L-PEI has been used to efficiently deliver genes in vivo into a wide range of organs such as lung, brain, pancreas, retina, bladder as well as tumor. L-PEI is able to efficiently condense, stabilize and deliver nucleic acids in vitro and in vivo.

The nucleic acids and vectors may also be applied to a surface of a device (e.g., a catheter) or contained within a pump, patch, or other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

In some embodiments of the invention, liposomes are used to effectuate transfection into a cell or tissue. The pharmacology of a liposomal formulation of nucleic acid is largely determined by the extent to which the nucleic acid is encapsulated inside the liposome bilayer. Encapsulated nucleic acid is protected from nuclease degradation, while those merely associated with the surface of the liposome is not protected. Encapsulated nucleic acid shares the extended circulation lifetime and biodistribution of the intact liposome, while those that are surface associated adopt the pharmacology of naked nucleic acid once they disassociate from the liposome. Nucleic acids may be entrapped within liposomes with conventional passive loading technologies, such as ethanol drop method (as in SALP), reverse-phase evaporation method, and ethanol dilution method (as in SNALP).

Liposomal delivery systems provide stable formulation, provide improved pharmacokinetics, and a degree of ‘passive’ or ‘physiological’ targeting to tissues. Encapsulation of hydrophilic and hydrophobic materials, such as potential chemotherapy agents, are known. See for example U.S. Pat. No. 5,466,468 to Schneider, which discloses parenterally administrable liposome formulation comprising synthetic lipids; U.S. Pat. No. 5,580,571, to Hostetler et al. which discloses nucleoside analogues conjugated to phospholipids; U.S. Pat. No. 5,626,869 to Nyqvist, which discloses pharmaceutical compositions wherein the pharmaceutically active compound is heparin or a fragment thereof contained in a defined lipid system comprising at least one amphipathic and polar lipid component and at least one nonpolar lipid component.

Liposomes and polymerosomes can contain a plurality of solutions and compounds. In certain embodiments, the complexes of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21(20):9183-6, incorporated by reference.

In some embodiments of the invention, non-viral vectors are modified to effectuate targeted delivery and transfection. PEGylation (i.e. modifying the surface with polyethyleneglycol) is the predominant method used to reduce the opsonization and aggregation of non-viral vectors and minimize the clearance by reticuloendothelial system, leading to a prolonged circulation lifetime after intravenous (i.v.) administration. PEGylated nanoparticles are therefore often referred as “stealth” nanoparticles. The nanoparticles that are not rapidly cleared from the circulation will have a chance to encounter infected cells.

In some embodiments of the invention, targeted controlled-release systems responding to the unique environments of tissues and external stimuli are utilized. Gold nanorods have strong absorption bands in the near-infrared region, and the absorbed light energy is then converted into heat by gold nanorods, the so-called “photothermal effect”. Because the near-infrared light can penetrate deeply into tissues, the surface of gold nanorod could be modified with nucleic acids for controlled release. When the modified gold nanorods are irradiated by near-infrared light, nucleic acids are released due to thermo-denaturation induced by the photothermal effect. The amount of nucleic acids released is dependent upon the power and exposure time of light irradiation.

Regardless of whether compositions are administered as nucleic acids or polypeptides, they are formulated in such a way as to promote uptake by the mammalian cell. Useful vector systems and formulations are described above. In some embodiments the vector can deliver the compositions to a specific cell type. The invention is not so limited however, and other methods of DNA delivery such as chemical transfection, using, for example calcium phosphate, DEAE dextran, liposomes, lipoplexes, surfactants, and perfluoro chemical liquids are also contemplated, as are physical delivery methods, such as electroporation, micro injection, ballistic particles, and “gene gun” systems.

In other embodiments, the compositions comprise a cell which has been transformed or transfected with one or more Cas/gRNA vectors. In some embodiments, the methods of the invention can be applied ex vivo. That is, a subject's cells can be removed from the body and treated with the compositions in culture to excise, for example, VZV sequences and the treated cells returned to the subject's body. The cell can be the subject's cells or they can be haplotype matched or a cell line. The cells can be irradiated to prevent replication. In some embodiments, the cells are human leukocyte antigen (HLA)-matched, autologous, cell lines, or combinations thereof. In other embodiments the cells can be a stem cell. For example, an embryonic stem cell or an artificial pluripotent stem cell (induced pluripotent stem cell (iPS cell)). Embryonic stem cells (ES cells) and artificial pluripotent stem cells (induced pluripotent stem cell, iPS cells) have been established from many animal species, including humans. These types of pluripotent stem cells would be the most useful source of cells for regenerative medicine because these cells are capable of differentiation into almost all of the organs by appropriate induction of their differentiation, with retaining their ability of actively dividing while maintaining their pluripotency. iPS cells, in particular, can be established from self-derived somatic cells, and therefore are not likely to cause ethical and social issues, in comparison with ES cells which are produced by destruction of embryos. Further, iPS cells, which are self-derived cell, make it possible to avoid rejection reactions, which are the biggest obstacle to regenerative medicine or transplantation therapy.

The isolated nucleic acids can be easily delivered to a subject by methods known in the art, for example, methods which deliver siRNA. In some aspects, the Cas may be a fragment wherein the active domains of the Cas molecule are included, thereby cutting down on the size of the molecule. Thus, the, Cas9/gRNA molecules can be used clinically, similar to the approaches taken by current gene therapy. In particular, a Cas9/multiplex gRNA stable expression stem cell or iPS cells for cell transplantation therapy as well as vaccination can be developed for use in subjects.

Transduced cells are prepared for reinfusion according to established methods. After a period of about 2-4 weeks in culture, the cells may number between 1×10⁶ and 1×10¹⁰. In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent. For administration, cells of the present invention can be administered at a rate determined by the LD₅₀ of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces that may include, but are not restricted to, bone marrow or adipose tissues.

Methods of Treatment

In certain embodiments, a method of eradicating a VZV genome in a cell or a subject, comprises contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a VZV genome.

In other embodiments, a method of inhibiting replication of a VZV in a cell or a subject, comprising contacting the cell or administering to the subject, a pharmaceutical composition comprising a therapeutically effective amount of an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a VZV genome.

The compositions and molecules embodied herein, to prevent and/or treat a Herpesviridae infection, may be used alone or in combination with conventional therapeutic regimens such as surgery, irradiation, chemotherapy, bone marrow transplantation (autologous, syngeneic, allogeneic or unrelated) and/or one or more other compounds. These compounds are intended to include but are not limited to all varieties of drugs, particularly antibacterial, antibiotic, antiviral, anti-mycotics, anti-inflammatory agents, antiproliferative and antineoplastic drugs and agents, and neurotropic, psychotropic and anticonvulsant drugs or agents and the like. The compounds may be administered under a metronomic regimen. As used herein, “metronomic” therapy refers to the administration of continuous low-doses of a therapeutic agent. Therapeutic agents can include, for example, chemotherapeutic agents such as, cyclophosphamide (CTX, 25 mg/kg/day, p.o.), taxanes (paclitaxel or docetaxel), busulfan, cisplatin, cyclophosphamide, methotrexate, daunorubicin, doxorubicin, melphalan, cladribine, vincristine, vinblastine, and chlorambucil.

The compositions of the present invention can be prepared in a variety of ways known to one of ordinary skill in the art. Regardless of their original source or the manner in which they are obtained, the compositions disclosed herein can be formulated in accordance with their use. For example, the nucleic acids and vectors described above can be formulated within compositions for application to cells in tissue culture or for administration to a patient or subject. Any of the pharmaceutical compositions of the invention can be formulated for use in the preparation of a medicament, and particular uses are indicated below in the context of treatment, e.g., the treatment of a subject having a VZV viral infection or at risk for contracting a VZV virus infection. When employed as pharmaceuticals, any of the nucleic acids and vectors can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

The pharmaceutical compositions may contain, as the active ingredient, nucleic acids and vectors described herein in combination with one or more pharmaceutically acceptable carriers. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

An effective amount of any composition provided herein can be administered to an individual in need of treatment. An effective amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Dosage, toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As described, a therapeutically effective amount of a composition (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

Kits

The compositions described herein can be packaged in suitable containers labeled, for example, for use as a therapy to treat a subject having a VZV infection, or a subject at risk of contracting for example, a VZV infection. The containers can include a composition comprising a nucleic acid sequence, e.g. an expression vector encoding a CRISPR-associated endonuclease, for example, a Cas9 endonuclease, and a guide RNA complementary to a target sequence in a VZV genome, or a vector encoding that nucleic acid, and one or more of a suitable stabilizer, carrier molecule, flavoring, and/or the like, as appropriate for the intended use. Accordingly, packaged products (e.g., sterile containers containing one or more of the compositions described herein and packaged for storage, shipment, or sale at concentrated or ready-to-use concentrations) and kits, including at least one composition of the invention, e.g., a nucleic acid sequence encoding a CRISPR-associated endonuclease, for example, a Cas9 endonuclease, and a guide RNA complementary to a target sequence in a VZV genome, or a vector encoding that nucleic acid and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, applicants do not admit any particular reference is “prior art” to their invention.

### EXAMPLES

### Example 1: Materials and Methods

VZV ORF63 gRNA design: The genomic sequence of VZV ORF63 (NC_001348.1) was obtained from the NCBI database. Three gRNAs, ORF63m1, ORF63m2 and ORF63m3, were designed and selected using available online tools (https://benchling.com/).

ORF63-stable TC620 oligodendroglioma cell line: The human oligodendroglioma cell line, TC620, was maintained in DMEM supplemented with 10% FBS. For selection, cells were transfected with a vector expressing ORF63 and 2 days after transfection they were selected using the above medium containing 1500 μg of G418 selector (Life Technologies) for 2 weeks in order to produce clonal derivatives of the TC620 cells.

Cloning of VZV ORF63 gRNAs in px260 plasmid: A pair of DNA oligonucleotides from each target sequence were designed in forward and reverse orientations based on published and recommended flanking sequences for pX260 vector (Addgene plasmids 42229). Each pair were annealed in a thermocycler, using 5 μl of each oligo at the concentration of 100 nM at 95° C. for 7 mins and ramped at 3% from 95° C. to 25° C. in the presence of 2 μl of T4 DNA ligase buffer and 9 μl of water in a total reaction volume of 20 Annealed oligo pairs were then cloned into the pX260 vector linearized at the BbsI restriction site. The insertion of the gRNAs was confirmed by sequencing. Plasmid preps were prepared using Plasmid Mini kit (Qiagen).

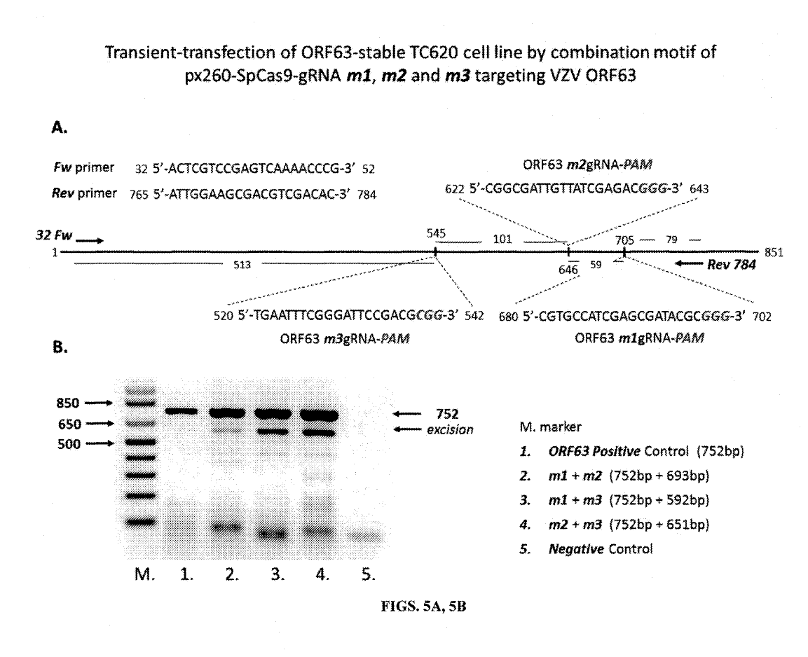

InDel mutation analysis: Deletions and/or insertions of nucleotides in VZV ORF63 were verified by co-transfection experiments in the ORF63-stable TC620 oligodendroglioma cell line. One microgram of each pX260 plasmid (carrying a human codon-optimized SpCas9 and one of the three ORF63-specific gRNAs, respectively) were co-transfected in combination. ORF63-stable TC620 oligodendroglioma cells transfected with empty vectors were served as control. Genomic DNA extractions were performed 48 h post-transfection using the NucleoSpin Tissue Kit (Machery-Nagel). Genomic DNA amplifications were performed using FailSafe PCR Enzyme (Epicentre) and two primers which were designed from the VZV ORF63 genomic sequence to amplify a fragment of 752 bp in size (as shown in FIGS. 5A, 5B) using following conditions: 94° C. (5 min) followed by 40 cycles of 94° C. (30s), 58° C. (30s), 72° C. (30s) and a final extension at 72° C. (4 min). The PCR products were analyzed on a 1.5% agarose gel.

What is claimed:

1. A composition for eradicating Herpesviridae in vitro or in vivo, the composition comprising: an isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Herpesviridae genome.

2. The composition of claim 1, wherein the Herpesviridae comprises: herpes simplex virus (HSV)-1, HSV-2, varicella zoster virus (VZV), human herpesvirus (HHV)-5 HHV-6, HHV-7, cytomegalovirus, Epstein Barr Virus, herpes zoster virus (HZ), equine herpesvirus 1 and 4, pseudorabies virus, bovine herpesvirus 1 and 5, HHV6A and HHV6B or herpes lymphotropic virus, HHV7 or Pityriasis Rosacea, SHV/HHV8 or simian varicella virus (herpes virus 9).

3. The composition of claim 2, wherein the Herpesviridae is varicella zoster virus (VZV) or a simian varicella virus (SVV).

4. The composition of claim 2, wherein the target nucleic acid sequence comprises one or more nucleic acid sequences in coding and non-coding nucleic acid sequences of the VZV or SVV genome.

5. The composition of claim 1, wherein the target nucleic acid sequence comprises one or more sequences within a sequence encoding structural proteins, non-structural proteins or combinations thereof.

6. The composition of claim 1, wherein a target nucleic acid sequence has at least a 75% sequence identity to nucleic acid sequences in unique long region (UL), terminal long (TRL) and internal long (IRL) repeats, unique short region (US), internal short repeats (IRS), terminal short repeats (TRS), open reading frames (ORF), glycoproteins, isomers or combinations thereof.

7. The composition of claim 1, wherein a target nucleic acid sequence comprises a nucleic acid sequence in unique long region (UL), terminal long (TRL) and internal long (IRL) repeats, unique short region (US), internal short repeats (IRS), terminal short repeats (TRS), open reading frames (ORF), glycoproteins, isomers or combinations thereof.

8. The composition of claim 1, wherein a target nucleic acid sequence comprises one or more nucleic acid sequences encoding an open reading frame (ORF) sequence, VZV glycoprotein E, VZV viral kinase ORF47, VZV viral kinase ORF66, VZV 1E62 protein, VZV 1E63 protein, VZV 1E70 protein, VZV IE71 protein, VZV DNA polymerase, and a VZV glycoprotein, ORF 63/70, ORF 62/71, ORF6, ORF28, ORF55, ORF25, ORF26, ORF30, ORF34, ORF 42/45, ORF 43, ORF54, ORF4, ORF5, ORF9A, ORF9, ORF 17, ORF20, ORF21, ORF22, ORF24, ORF27, ORF29, ORF 31, ORF33, ORF33.5, ORF37, ORF38, ORF39, ORF40, ORF41, ORF44, ORF46, ORF48, ORF50, ORF51, ORF52, ORF53, ORF56, ORF60, ORF61, ORF62, ORF64, ORF65, ORF66, ORF67, ORF68, and/or ORF69.

9. The composition of claim 1, wherein nucleic acid sequences comprising a gRNA sequence has at least a 75% sequence identity to nucleic acid sequences comprising SEQ ID NOS: 1-10, or combinations thereof.

10. The composition of claim 9, wherein the nucleic acid sequences comprising the gRNA sequences comprise: SEQ ID NO: 1-10, or combinations thereof.

11. The composition of claim 1, further comprising a short proto-spacer adjacent motif (PAM)-presenting DNA oligonucleotide sequence.

12. An isolated nucleic acid sequence encoding a Clustered Regularly Interspaced Short Palindromic Repeat (CRISPR)-associated endonuclease and at least one guide RNA (gRNA), the gRNA being complementary to a target nucleic acid sequence in a Varicella zoster virus (VZV) genome.

13. The isolated nucleic acid sequence of claim 12, wherein the target nucleic acid sequence comprises one or more sequences within a sequence encoding structural proteins, non-structural proteins or combinations thereof.

14. The isolated nucleic acid sequence of claim 12, wherein a target nucleic acid sequence has at least a 75% sequence identity to nucleic acid sequences in unique long region (UL), terminal long (TRL) and internal long (IRL) repeats, unique short region (US), internal short repeats (IRS), terminal short repeats (TRS), open reading frames (ORF), glycoproteins, isomers or combinations thereof.

15. The isolated nucleic acid sequence of claim 12, wherein a target nucleic acid sequence comprises a nucleic acid sequence in unique long region (UL), terminal long (TRL) and internal long (IRL) repeats, unique short region (US), internal short repeats (IRS), terminal short repeats (TRS), isomers or combinations thereof.

16. The isolated nucleic acid sequence of claim 12, wherein a target nucleic acid sequence comprises one or more nucleic acid sequences encoding an open reading frame (ORF) sequence, VZV glycoprotein E, VZV viral kinase ORF47, VZV viral kinase ORF66, VZV 1E62 protein, VZV 1E63 protein, VZV 1E70 protein, VZV IE71 protein, VZV DNA polymerase, and a VZV glycoprotein, ORF 63/70, ORF 62/71, ORF6, ORF28, ORF55, ORF25, ORF26, ORF30, ORF34, ORF 42/45, ORF 43, ORF54, ORF4, ORF5, ORF9A, ORF9, ORF 17, ORF20, ORF21, ORF22, ORF24, ORF27, ORF29, ORF 31, ORF33, ORF33.5, ORF37, ORF38, ORF39, ORF40, ORF41, ORF44, ORF46, ORF48, ORF50, ORF51, ORF52, ORF53, ORF56, ORF60, ORF61, ORF62, ORF64, ORF65, ORF66, ORF67, ORF68, and/or ORF69.

17. An isolated nucleic acid sequence encoding a nuclease; and a sequence-specific targeting moiety that targets the nuclease to a Herpesviridae nucleic acid in vivo within a host cell thereby causing the nuclease to cleave the Herpesviridae nucleic acid without interfering with host nucleic acid.

18. The isolated nucleic acid sequence of claim 17, wherein the nuclease is a Cas9 endonuclease and the sequence-specific binding module comprises at least one guide RNA that specifically targets a portion of a viral genome.

19. The isolated nucleic acid sequence of claim 17, wherein the Cas9 endonuclease and the guide RNA are co-expressed in a host cell infected by a Herpesviridae.

20. The isolated nucleic acid sequence of claim 17, wherein the Herpesviridae is Varicella zoster virus (VZV).

21. The isolated nucleic acid sequence of claim 20, wherein the at least one guide RNA is complementary to a portion of a VZV genome.

22. The isolated nucleic acid sequence of claim 20, wherein one or more guide RNA are designed to target the nuclease to cleave the VZV genome within one or more sequences encoding a gene product that is necessary for VZV replication and/or function.

23. The isolated nucleic acid sequence of claim 22, wherein one or more sequences encoding a gene product that is necessary for VZV replication and/or function comprise: a replication origin, a terminal repeat, a replication factor binding site, a promoter, a coding sequence, a non-coding sequence, or a repetitive region.

24-29. (canceled)


SEQUENCE LISTING (15 sequences)

SEQ ID NO: 1 - DNA, 25 bp, Artificial Sequence
  Description of Artificial Sequence Synthetic oligonucleotide
caccgtgaatttcgggattccgacg

SEQ ID NO: 2 - DNA, 25 bp, Artificial Sequence
  Description of Artificial Sequence Synthetic oligonucleotide
aaaccgtcggaatcccgaaattcac

SEQ ID NO: 3 - DNA, 25 bp, Artificial Sequence
  Description of Artificial Sequence Synthetic oligonucleotide
caccgatacgcgggtgcagaaaccg

SEQ ID NO: 4 - DNA, 25 bp, Artificial Sequence
  Description of Artificial Sequence Synthetic oligonucleotide
aaaccggtttctgcacccgcgtatc

SEQ ID NO: 5 - DNA, 23 bp, Artificial Sequence
  Description of Artificial Sequence Synthetic oligonucleotide
cgtgccatcgagcgatacgcggg

SEQ ID NO: 6 - DNA, 23 bp, Artificial Sequence
  Description of Artificial Sequence Synthetic oligonucleotide
cccgcgtatcgctcgatggcacg

SEQ ID NO: 7 - DNA, 22 bp, Artificial Sequence
  Description of Artificial Sequence Synthetic oligonucleotide
cggcgattgttatcgagacggg

SEQ ID NO: 8 - DNA, 22 bp, Artificial Sequence
  Description of Artificial Sequence Synthetic oligonucleotide
cccgtctcgataacaatcgccg

SEQ ID NO: 9 - DNA, 23 bp, Artificial Sequence
  Description of Artificial Sequence Synthetic oligonucleotide
tgaatttcgggattccgacgcgg

SEQ ID NO: 10 - DNA, 23 bp, Artificial Sequence
  Description of Artificial Sequence Synthetic oligonucleotide
ccgcgtcggaatcccgaaattac

SEQ ID NO: 11 - DNA, 34 bp, Varicella-zoster virus
gatgtaattgaatttcgggattccgacgcggaat

SEQ ID NO: 12 - DNA, 34 bp, Varicella-zoster virus
atcgagcgatacgcgggtgcagaaaccgcggaat

SEQ ID NO: 13 - DNA, 829 bp, Varicella-zoster virus
atgttttgcacctcaccggctacgcggggcgactcgtccgagtcaaaacccggggcatcggttgatgttaacggaaagatggaatatggatctgcaccaggacccccgggatacgtcgcggggccccggcgcgttttgtactccgggttgggagatccacccggccaggctcgttgaggacatcaaccgtgtttttttatgtattgcacagtcgtcgggacgcgtcacgcgagattcacgaagattgcggcgcatatgcctcgacttttatctaatgggtcgcaccagacagcgtcccacgttagcgtgctgggaggaattgttacagcttcaacccacccagacgcagtgcttacgcgctactttaatggaagtgtcccatcgaccccctcggggggaagacgggttcattgaggcgccgaatgttcctttgcataggagcgcactggaatgtgacgtatctgatgatggtggtgaagacgatagcgacgatgatgggtctacgccatcggatgtaattgaatttcgggattccgacgcggaatcatcggacggggaagactttatagtggaagaagaatcagaggagagcaccgattcttgtgaaccagacggggtacccggcgattgttatcgagacggggatgggtgcaacaccccgtccccaaagagaccccagcgtgccatcgagcgatacgcgggtgcagaaaccgcggaatatacagccgcgaaagcgctcaccgcgttgggcgaggggggtgtagattggaagcgacgtcgacacgaagccccgcgccggcatgatataccgcccccccatggcgtgtag

SEQ ID NO: 14 - DNA, 21 bp, Artificial Sequence
  Description of Artificial Sequence Synthetic primer
actcgtccgagtcaaaacccg

SEQ ID NO: 15 - DNA, 20 bp, Artificial Sequence
  Description of Artificial Sequence Synthetic primer
attggaagcgacgtcgacac